(19) United States
(12) Patent Application Publication
(10) Pub. No.: US 2024/0212799 A1
(43) Pub. Date: Jun. 27, 2024

(54) SYSTEMS AND METHODS FOR IMPROVING WELLBEING THROUGH THE GENERATION OF PERSONALISED APP RECOMMENDATIONS

(71) Applicant: SYNDI LTD, Cambridge, Cambridgeshire (GB)

(72) Inventors: Jorge ALEXANDER, Cambridge, Cambridgeshire (GB); Benjamin LAKEY, Cambridge, Cambridgeshire (GB)

(21) Appl. No.: 18/288,263

(22) PCT Filed: Apr. 19, 2022
(86) PCT No.: PCT/IB2022/053642

§ 371 Date: Oct. 25, 2023

(30) Foreign Application Priority Data
Apr. 20, 2021 (GB) 2105656.9

(51) Int. Cl. G16H 10/20; G16H 20/00; G16H 50/30

(52) U.S. Cl. CPC G16H 10/20; G16H 20/00; G16H 50/30

(57) ABSTRACT

Systems and methods for recommending an application for the improvement of wellbeing are disclosed. The methods and systems comprise sending a first survey to a user's device, receiving usage statistics of a plurality of applications on the user's device, receiving the result data of the first survey, and recommending an application based on the result data and usage statistics.

### BACKGROUND

The present disclosure relates to application recommendation systems and methods and, more particularly, to systems and methods for recommending applications based on a user's current application usage and survey result data to improve the user's wellbeing.

### SUMMARY

Over the past 30 years, successive governments across the world have pledged to improve health in their jurisdictions and to address barriers that create inequalities in health. Mental health has been a key priority area and featured strongly in policy in recent years. Health policy including, for example, mental health and wellbeing, is complex, and patient and user support is episodic. Early intervention and support isn't possible when patients and clinicians aren't aware of changes in the health and wellness of patients and users.

Many people with mental illnesses or mental health issues don't receive any professional help. Assessments are resource-intensive and lack continuous feedback and improvement. Questionnaires require high patient motivation and engagement. Those with the most severe cases of mental illnesses will, generally, get professional help, but it can take years before they are correctly diagnosed and receive effective treatment.

Like any illness, reducing the time for correct diagnosis and obtaining effective treatment is vital in the care of a patient or user. Often, care is provider-focused and is not personalized to every individual. In the early stages of care and treatment, signposting to support resources and apps is often suggested by mental health first aiders or support workers, but they don't take into account each user's needs.

The World Health Organization (WHO) defines good health as good social, mental, and physical wellbeing. The WHO has gone on to state that mental illness can be a major cause of long-term disability worldwide. It is widely accepted that mental illness ranks highly on the scale of disease burden in developed countries, alongside cardiovascular diseases, cancers, and musculoskeletal disorders. Diseases burden is the combined effect of premature death and years lived with disability caused by an illness.

Mental health issues often start in adolescence or early adulthood. When mental illnesses start at this stage in life, they can affect the young person's education, movement into adult occupational roles, forming of key social relationships including marriage, and increase the likelihood of alcohol or other drug misuses. Consequently, mental illnesses can cause disability across a person's lifespan. Therefore, there is a need to detect problems early and ensure an individual receives appropriate treatment and support.

Accordingly, there is a need for an application (App) recommendation system and methods, such as those described herein. The systems and methods are introduced to provide an improvement to the wellbeing of the user through the generation of personalized application recommendations, based on a perceived impact score of applications on the user's device, which, during the survey period, affect the result data of the survey. In particular, systems and methods described herein provide personalized signposting to support resources and digital tools through intelligent assessment and monitoring of a user.

In one approach, there is provided a method for providing an application recommendation, wherein the method comprises sending a first survey to a user's device, receiving usage statistics of a plurality of applications on the user's device, receiving result data of the first survey, and recommending an application based on the result data and usage statistics.

In some examples, the method further comprises calculating an impact score for each application of the plurality of applications. In some examples, the impact score represents an impact that each application of the plurality of applications has on the survey result data.

In some examples, the method further comprises building an impact matrix based on the calculated impact score. In some examples, the impact matrix represents an average impact of each application of the plurality of applications.

In some examples, the method further comprises sending the calculated impact score and impact matrix to a data storage. In some examples, the method further comprises processing the usage statistics and survey result data with the impact matrix to generate the recommendation of an application.

In some examples, the method further comprises initiating a wait period. In some examples, the wait period is initiated in response to one of: the survey being completed, the result data being sent to data storage; or in response to the user not opening the first survey.

In some examples, the method further comprises sending a second survey. In some examples, the second survey is sent after the wait period ends. In some examples, the first and second surveys are accessed through a URL sent to the user device.

In some examples, the method further comprises requesting permission to access app usage statistics. In addition, in response to being granted permission, the method further comprises sending app usage statistics to data storage. Whereas, in response to being denied permission, the method further comprises waiting for permission to be granted before accessing app usage statistics.

In another approach, a system for providing an application recommendation is provided. The system comprises data storage, a survey delivery apparatus, a user device, and control circuitry configured to carry out the method as described above, and in more detail below.

In another approach, anon-transitory machine-readable medium comprising memory with instructions encoded thereon for providing an application recommendation is provided. When executed, the non-transitory machine-readable medium instructions carry out the method as described above, and in more detail below.

In a final approach, an apparatus for providing an application recommendation is provided. The apparatus comprises means suitable for carrying out the method as described above, and in more detail below.

In this way, the present disclosure enables deeper personalization in addition to decreased engagement requirements by utilizing the most accessible and rich window into a user's health and behavior—device data, such as smartphone data. Syndi's recommendation platform helps users find the right support for them, without asking for overwhelming amounts of information.

The advantages of the present disclosure will be apparent upon review. However, by way of summary, methods, and systems enable a user to measure and self-manage their health—without asking for overwhelming amounts of information, provide personalized recommendations based on the user's requirements and engagement preferences, provide proactive digital support for a user's health, measure changes and patterns in clinical scores and behavior of a user of a device. In addition, the present disclosure allows a user to increase their self-management and awareness of their health and wellbeing.

The methods and systems described in more detail below may refer to mental health and wellbeing. However, it should be noted that the methods and systems are not limited to mental health and wellbeing, and may also include chronic pain, musculoskeletal conditions, diabetes management, and birth control management, for example. Indeed, any ailments, medicaments or rehabilitation that requires self-management and awareness would benefit from the advantages and disclosures herein.

### BRIEF DESCRIPTION OF THE DRAWINGS

The above and other objects and advantages of the disclosures will be apparent upon consideration of the following detailed description, taken in conjunction with the accompanying drawings, in which:

FIG. 1 is a flowchart of illustrative steps involved in recommending an App, in accordance with systems, methods, and embodiments of the present disclosure.

FIG. 2 illustrates a block diagram representing devices, components of each device, and data flow therebetween for an exemplary system, in accordance with systems, methods, and embodiments of the present disclosure.

FIG. 3 is an exemplary survey comprising a plurality of questions to be provided to a user in a clinical assessment.

FIG. 4 is a flowchart of illustrative steps involved in the delivery and response collection of a clinical survey, in accordance with systems, methods, and embodiments of the present disclosure.

FIG. 5 is a flowchart of illustrative processing steps involved in the collection of App usage statistics.

FIG. 6 is a flowchart of illustrative processing steps involved in the building of an impact matrix, in accordance with systems, methods, and embodiments of the present disclosure.

FIG. 7 is a flowchart of illustrative processing steps involved in the selecting of an App recommendation of apps for the improvement of mental wellbeing.

FIGS. 8A to 8D depicts exemplary tables of results of the processing, data collection, surveys, and impact score for Apps in accordance with systems, methods, and embodiments of the present disclosure.

FIG. 9 is a block diagram representing devices, components of each device, and data flow therebetween for an app recommendation system, in accordance with some methods and embodiments of the present disclosure.

### DETAILED DESCRIPTION

Methods and systems are provided herein for improving the mental wellbeing of a user of a device.

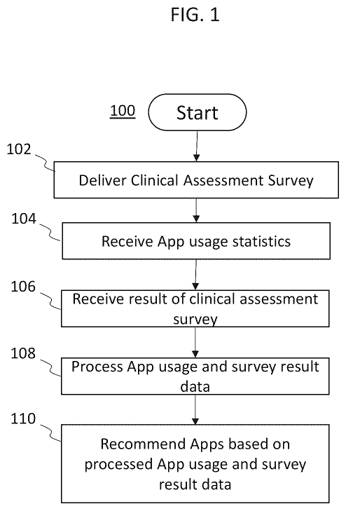

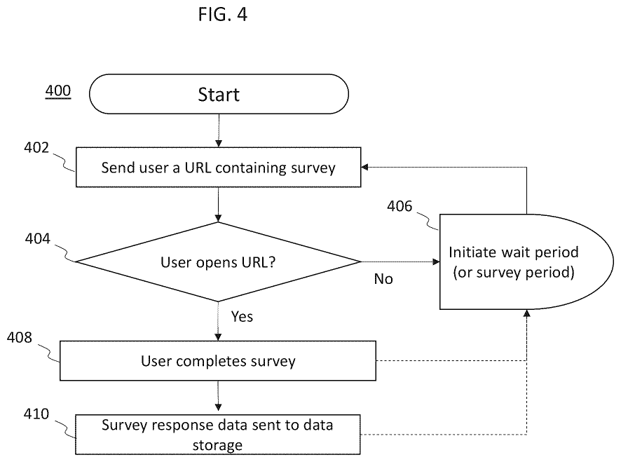

FIG. 1 is a flowchart of illustrative steps involved in recommending an App, in accordance with systems, methods, and embodiments of the present disclosure. Process 100 begins at step 102 wherein a clinical assessment survey is delivered to the user. The method of delivering the survey can be either by an SMS, email, or any other electronic means containing a URL to the survey, or to an executable application to be installed on the user's device. More detail regarding the process for sending the survey to a user is described with reference to FIG. 4, below.

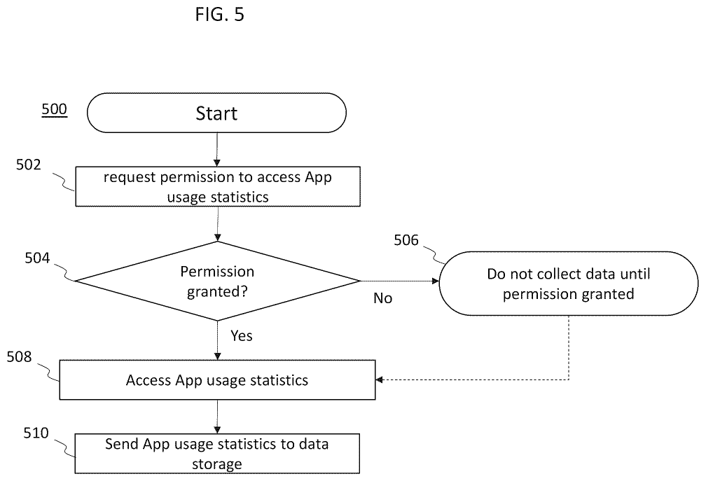

At step 104, the system receives App usage statistics. In some examples, the App usage statistics for a plurality of applications running on the user's device are obtained after the user grants permission. The information contained within the App usage statistics may comprise a total usage over a given period, how often a given application is accessed, the time of day an application is accessed, and the like. More detail regarding the process for requesting permission to access App usage statistics is described with reference to FIG. 5, below. For example, the App usage statistic may already be stored in data storage, and received therefrom, or may be transmitted directly from the user's device. In some examples, a combination of transmission from the user's device and historic data from the data storage may be used in the methods and systems described herein.

At step 106, the result of the clinical assessment survey is received by the system, referred to as result data. In some examples, the clinical assessment survey may be received from a data storage. In some examples, the result data may be transmitted directly from a user's device. In other examples, a combination of result data from the user's device and historic data from data storage may be used in the method and systems described here.

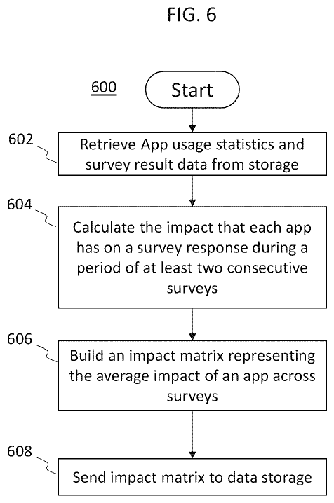

At step 108, App usage statistics and survey result data are processed. In some examples, the App usage statistics and survey result data are processed to determine an impact score of each of a plurality of applications on the user's device on the questions of the survey in a survey period. Thereafter, in some examples, the average impact of each application across a plurality of surveys is determined by building an impact matrix. More detail on how the impact score and the impact matrix are determined is described with reference to FIG. 6, below.

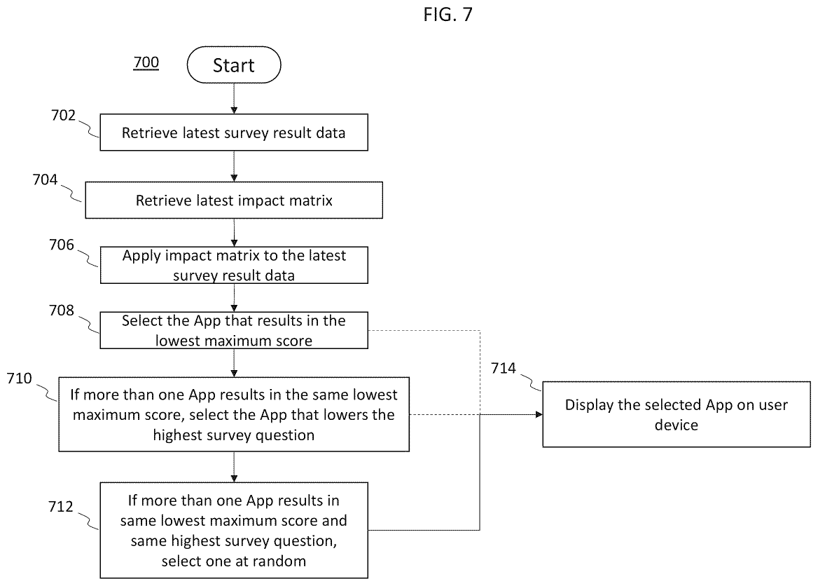

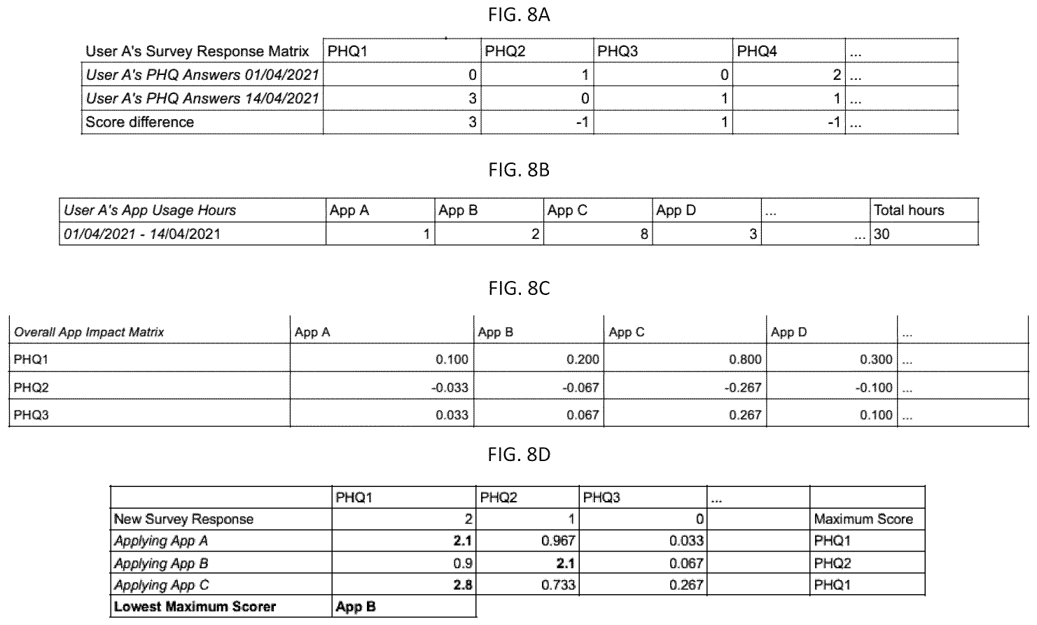

At step 110, Apps based on processed App usage and survey result data are recommended. Apps that result in the lowest maximum impact score are chosen to be recommended to the user. If more than one App results in the same lowest maximum score, then the App that lowers the highest survey question is chosen. If more than one App results in the same lowest maximum score and same highest survey question, then an App is selected at random. More detail on the recommendation of Apps is discussed with reference to FIGS. 7 and 8, below.

Accordingly, as will be discussed below, in some examples, there is provided a system for mental wellbeing improvement through the generation of personalized application recommendations comprising of a clinical assessment survey delivery system configured to deliver, process, and store, via interfacing with a data store, clinical surveys to measure a user's mental health, comprising of an SMS delivery system and online survey completion form.

In some examples, the clinical assessment survey delivery system may take the form of a smartphone application configured to access, process, and send statistics of a user's App usage statistics during the period between successive survey completion.

In some examples, a data storage configured to store the collected statistics of app usage statistics and survey result data is connected to all entities of the systems described herein. In some examples, the data storage is configured to store historic App usage statistics and survey result data, or impact scores and matrices that reflect the App usage statistics and survey result data.

In some examples, there is an application recommendation system configured to interface with a data store and comprising of a processing engine configured to: calculate the impact that an app has when applied to a set of survey questions; calculate the effect of an app on a user's most recent survey response; select the app that minimizes the highest scoring answer of the user's most recent survey response; display the selected app to the user.

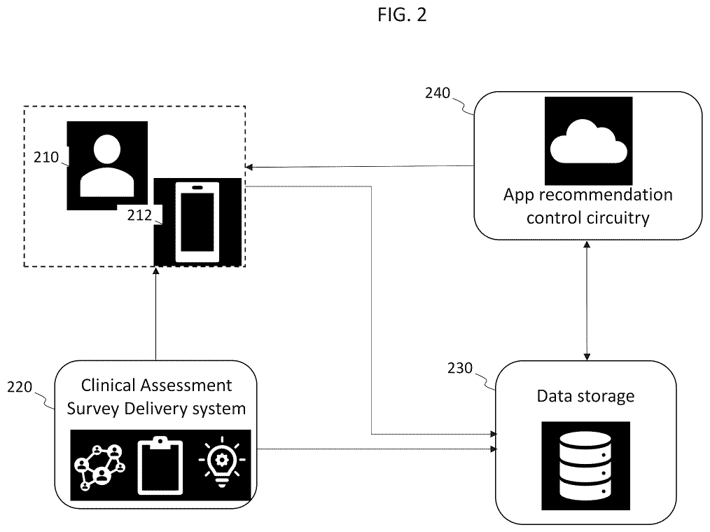

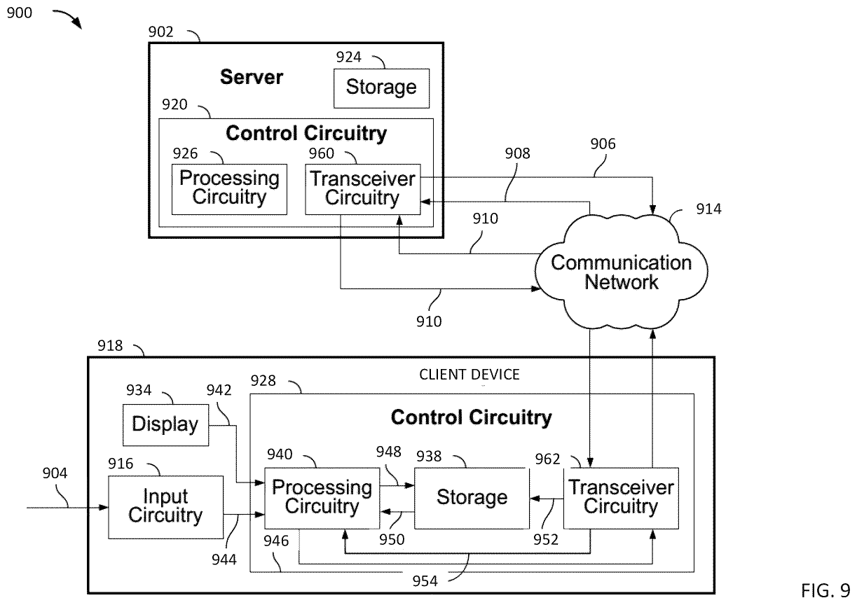

FIG. 2. depicts a block diagram representing devices, components of each device, and data flow therebetween for an exemplary system, in accordance with systems, methods, and embodiments of the disclosure. Shown is a user 210 and a user device 212. The user device 212 may be a smartphone, as shown, or any other suitable client device, such as those discussed in more detail with reference to FIG. 9, e.g., client device 918.

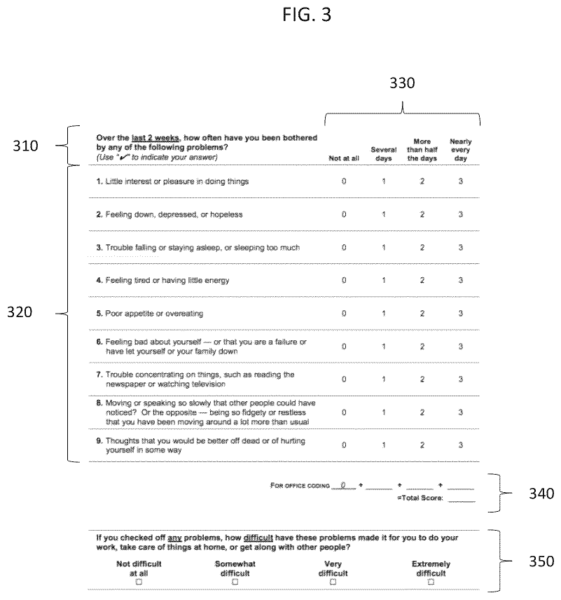

Generally, the clinical assessment survey delivery system 220 provides a survey to the user 210 by sending a URL via electronic communication to user device 212. The URL may be sent via SMS message, email, or any other electronic means such as, for example, MMS, WiFi®, Bluetooth®, etc. The clinical assessment survey delivery system 220 may provide a survey such as a survey shown and described with regard to FIG. 3. After user 210 fills in the survey via user device 212, the survey result data is transmitted to data storage 230 for storage. The clinical assessment survey delivery system 220 may also transmit the survey before user 210 completes the survey to the data storage 230.

The user device 212 may also transmit application usage statistics (App usage statistics) to data storage 230. The App usage statistics comprise the usage of a plurality of applications on the user device 212 during a survey period. In some examples, the survey period is defined as the time between a first and a second survey. The survey period may also be referred to as a wait period, which is initiated in response to one of the following events: the first survey being completed, the first result data being sent to data storage, or in response to the user not opening the first survey. In some examples, the survey period begins after the wait period ends. The wait period may be configurable, such as not to overwhelm the user 210 with too many surveys in a short period.

In some examples, Data storage 230 comprises control circuitry (not shown) to carry out processing on the survey result data and the App usage statistics. In other examples, the data storage 230 transmits the survey result data and the App usage statistics to App recommendation control circuitry 240 for further processing.

In some examples, the processing carried out on App usage statistics and survey result data comprises generating an impact score of each application of the plurality of applications on user device 212. In some examples, the impact score is defined by the equation:

$${I}_{asq} = \frac{{H}_{a s}}{{\sum }_{a} {H}_{as}} {Δ}_{sq}$$

where I_{asq} represents the impact score of an application, a, on a question, q, during a survey period, s;
where Hₐₛ represents hours of usage of the application, a, during the survey period, s;
where Δ_{sq} represents a change in a survey question score during the survey period, s.

The impact score wherein the impact score represents an impact that each application of the plurality of applications has on the survey result data. For example, any given application, such as application A, may impact the result of any given question, such as question Q, more so than application B. In such an example, application B may be recommended to the user. More detail on the impact score, and how the applications on the user device 212 will be discussed in more detail below.

Any of the devices, components, and data flow therebetween as described above may be considered separate entities. However, in some examples, the clinical assessment survey delivery system 220, the data storage 230, and App recommendation control circuitry 240 may be regarded as a single entity such as an apparatus or a system configured to carry out the embodiments of the present disclosure. In other examples, the survey delivery system 220, the data storage 230, and App recommendation control circuitry 240 may all be cloud-based or a mixture of cloud-based entities and local apparatuses.

FIG. 3 is an exemplary survey comprising a number of questions to be provided to the user 210 in a clinical assessment. In some examples, the survey shown in FIG. 3 is sent to user 210 by clinical assessment survey delivery system 220, as described with reference to FIG. 2, above. The survey comprises a header 310 which comprises instructions for the user 210 to understand how to answer the questions within the survey.

In more detail, header 310 may comprise an overall question for the user 210 to consider while answering the survey questions, such as “over the last 2 weeks, how often have you been bothered by any of the following problems?”. The survey questions (shown as questions 1 to 9, however, there may be more or fewer questions for the user to answer), may then comprise statements 320 which the user can score using the numeric scoring system 330. For example, the user may consider that they have been feeling tired or having little energy nearly every day over the last 2 weeks, in which case the user would tick “3” on the numeric scoring system 330 for statement number 4.

In some examples, the numeric scoring system 330 comprises discrete values 0 to 3, as shown in FIG. 3. However, it should be understood that the numeric scoring system 330 may comprise higher value numbers, above 3, and lower value numbers such as a negative score, for example, −2 or −1. The total of each score is added in the total scoring portion 340 of the survey. The total score of the numeric scoring system 330 is used in the calculation of the impact score.

In some examples, the survey comprises an additional question in an additional question portion 350. The additional question may also comprise a header 310, statements 320, numeric scoring system 330, and total scoring portion 340. In some examples, the additional question portion 350 comprises a relatively subjective question that allows the user to express how they are feeling without having to quantify their feelings with a number, as shown. In some examples, the calculated impact score will also take a total score or other data from the additional question portion 350.

In some examples, a high score indicates an urgent need for clinical intervention. For example, the systems and methods may label a user's profile, or a user's survey response data for clinical intervention. In some examples, a high score would have to pass a threshold to enable a clinical intervention. The threshold may be based on an abnormally high response for a particular user (i.e. a high standard deviation, which indicates a result far from a user's mean result). In some examples, a high score may be determined by a clinician on a per user basis, or a per questionnaire basis. For example, any particular questionnaire may be determined to result in a high score for any results above ‘0’, or any other arbitrarily chosen value.

In some examples, a high score may initiate an automated response that recommends that directs the user to “Emergency Resources”. For example, an emergency response procedure may be initiated that provides the user with crisis line information such as: a SHOUT text message crisis line, Samaritans Crisis line, NHS non-emergency service 111 and/or emergency services such as 999 or 911.

In some examples, the systems and methods disclosed here the questionnaire may begin with a choice between ‘Mind or Body’ before presenting the user with a questionnaire targeted at those specific ailments. For examples, upon choosing ‘Mind or Body’, a specific questionnaire aimed at the separate pathway will be presented to the user. As described with reference to the summary, there are strong correlations between physical illnesses and disabilities with mental illnesses so over time; therefore the questionnaire and examples herein are not limited to separate pathways and each pathway may comprise questions from another pathway, and a future questionnaire may comprise more integration between said pathways.

FIG. 4 is a flowchart of illustrative steps involved in the delivery and response collection of a clinical survey, in accordance with systems, methods, and embodiments of the present disclosure. Process 400 begins at step 402, which starts with sending the user 210 a URL containing the survey, such as the survey as described with reference to FIG. 3. The URL may be sent in any suitable electronic message or format, such as an SMS, MMS, email, or the like. In some examples, the URL as described herein may lead to a clinical application containing the survey. If the user's device 212 already has installed on it the clinical application the URL will open the survey within the clinical application.

At step 404, it is established whether or not user 210 has opened the URL containing the survey. If the user does not open the URL, the process continues to step 406. At step 406, a wait period may be initiated. The wait period is a configurable amount of time that will elapse before the process returns to step 402. In some examples, the wait period may also be called a survey period, that is to say, that the wait period may establish the period between surveys. In other examples, the wait period may directly precede the survey period (not shown).

If the user does open the URL the process continues to step 408. At step 408, user 210 completes the survey. The results of the survey are known as the result data. After the completion of the survey by user 210, the process continues to step 410. At step 410, the result data is sent to data storage, such as data storage 230. As shown in FIG. 4, the wait period or survey period may be initiated in parallel to step 408 or 410.

FIG. 5 is a flowchart of illustrative processing steps involved in the collection of App usage statistics. Process 500 may be carried out by an executable application on the user's device 212, such as a clinical application installed on the user's device 212, as described above.

Process 500 begins at step 502. Step 502 describes requesting permission to access App usage statistics on the user's device 212. At step 504, it is decided whether or not permission to access the App usage statistics has been granted.

If the permission has not been granted, process 500 continues to step 506. At step 506, no data is collected until permission is granted by user 210. In this way, the personal data of user 210 is kept on the user's device 212 until a time in which the user permits the data to be collected. If the permission has been granted, process 500 continues to step 508. At step 508, the App usage statistics of the user's device 212 are accessed.

At step 510, the App usage statistics are sent to data storage, such as data storage 230. The communication of the App usage statistics to the data storage 230 is sent by any suitable communication means or methods, such as via SMS, MMS, email, or via encrypted communication over the internet, such as those described in more detail with reference to FIG. 9 below.

FIG. 6 is a flowchart of illustrative processing steps involved in the building of an impact matrix, in accordance with systems, methods, and embodiments of the present disclosure. Process 600 begins at step 602, which described that the App usage statistics and survey result data are retrieved from data storage, such as data storage 230. The App usage statistics may comprise hours of usage of a plurality of applications on the user's device 212. The survey result data comprises the numeric scoring system for the questions and statements as described with regard to FIG. 3, above.

At step 604, process 600 involves calculating the impact that each App on the user device 212 has on survey response. In some examples, the impact score is defined by the equation:

$${I}_{asq} = \frac{{H}_{a s}}{{\sum }_{a} {H}_{as}} {Δ}_{sq}$$

where I_{asq} represents the impact score of an application, a, on a question, q, during a survey period, s;
where Hₐₛ represents hours of usage of the application, a, during the survey period, s;
where Δ_{sq} represents a change in a survey question score during the survey period, s.

In some examples, the impact score may only be generated during a period of at least two consecutive surveys. In these examples, the first survey is a “seed” survey, designed to establish a baseline result that will be used to compare against a second survey to establish the change in a survey question score, Asq.

In some examples, the impact score represents an impact that each application of the plurality of applications has on the survey result data. For example, any given application, such as application A, may impact the result of any given question, such as question Q, more so than application B. In such an example, application B may be recommended to the user.

At step 606, process 600 comprises building an impact matrix representing the average impact of a given application, such as application A, across at least two surveys. That is to say that the impact matrix represents an average impact of each application of the plurality of applications. In some examples, the impact matrix is defined by:

$${I}_{aq} = \frac{1}{S} {\sum }_{S = 1}^{S} {I}_{asq}$$

where S represents a total number of surveys
where I_{aq} represents an impact of an application, a, on a question, q.

At step 608, process 600 comprises sending the impact matrix to a data storage, such as data storage 230. In some examples, the impact matrix may be calculated by processing circuitry within the data storage (not shown) in which case step 608 may be omitted, or may comprise sending the impact matrix to another entity in the system as described herein.

The application of the impact score and the impact matrix to the result data and the App usage statistics results in an App recommendation to the user. The App recommendation seeks to improve their mental wellbeing by monitoring the impact each of the plurality of applications has on the numeric score the user assigns to the statements 320 in between successive surveys.

FIG. 7 is a flowchart of illustrative processing steps involved in the recommendation of apps for the improvement of the mental wellbeing of the user. Process 700 begins at step 702, which describes retrieving the latest survey result data. The latest survey result data may be retrieved from data storage, such as data storage 230, or received directly from user device 212. At step 704, the latest impact matrix is retrieved. The latest impact matrix may also be retrieved from data storage, such as data storage 230, or received directly from user device 212.

At step 706, the impact matrix is applied to the latest survey result data. More detail on the form and result of applying the impact matrix to the survey result is described with reference to FIG. 8, below.

At step 708, the App that results in the lowest maximum impact score is selected. If an App is selected at this stage, process 700 may continue to step 714. At step 714, the selected App is displayed on the user device. If no App can be select at this stage because, for example, more than one App results in the same lowest maximum impact score, then the process 700 continues on to step 710.

At step 710, the App that lowers the highest numeric score in the latest survey result data is selected. If an App is selected at this stage, process 700 may continue to step 714. If no App can be select at this stage because, for example, more than one App results in the same lowest maximum impact score and a same highest survey question, then the process 700 continues on to step 712. At step 712, an App is selected at random.

The steps described above (i.e. steps 702-714) may be performed by App recommendation control circuitry 240, or by control circuitry in the user device 212 (not shown) or data storage 230 (not shown).

It should be noted, and would be apparent to a person skilled in the art, the App recommendation includes, but is not limited to mental health and wellbeing, and includes, for example, other conditions such as chronic pain, musculoskeletal conditions, diabetes management, or birth control management.

FIGS. 8A to 8D depicts exemplary tables of results of the processing, data collection, surveys, and impact score for Apps in accordance with systems, methods, and embodiments of the present disclosure. FIG. 8A depicts the result data for two successive surveys for User A, taken on Apr. 1, 2021 and Apr. 14, 2021. In particular, there is shown a score difference for each of Patient Health Question 1 through 4 and so on. As shown. User A scored a 0 for question 1 on Apr. 1, 2021 but scored the same question with a 3 on Apr. 4, 2021, resulting in a score difference between the two successive surveys of 3. Similarly, User A scored a 1 for question 2 on Apr. 1, 2021 and a 0 for the same question on Apr. 14, 2021 resulting in a score difference between the two successive surveys of −1. In this example, the survey period would be considered the period between Apr. 1, 2021 and Apr. 14, 2021.

FIG. 8B depicts the App usage statistics during the survey period for the same User A. In particular, the App usage statistics describe that during the survey period Apr. 1, 2021 to Apr. 14, 2021. User A spent 1 hour on App A, 2 hours on App B, and so on, as shown in FIG. 8B. Also shown is a total of 30 hours of App usage during the survey period. In this example, the only App usage statistics shown are the hours of each App usage, but other data such as more granular total usage over the survey period, how often a given application is accessed, the time of day an application is accessed, and the like may be retrieved, monitored or measured.

FIG. 8C depicts the impact matrix each of a plurality of Apps A-D have had on the survey result data for User A based on the App usage statistics over the survey period of Apr. 1, 2021 and Apr. 14, 2021. In particular, FIG. 8C shows that App C has the largest positive impact matrix score for question 1 of the survey, but the largest negative impact matrix scored for question 2 of the survey. Other Apps, such as App A and B have varying impact matrix scores on the questions.

FIG. 8D depicts an applied impact matrix to a latest survey result data (shown as new survey response). As shown, based on the latest numeric score of 2 for question 1, after applying App A, a predicted score of 2.1 is generated; after applying App B, a predicted score of 0.9 is generated; after applying App C, a predicted score of 2.8 is generated. In particular, applying App B to the new score of 1 for question 2 results in a predicted score of 2.1, the same result as the predicted score after applying App A. The lowest maximum score is, therefore, the same after applying App A and App B to questions 1 and 2 respectively. In this scenario, the App that lowers the highest survey question is selected, so App B is selected (as was described with reference to FIG. 7, above). Thus an App has been selected based on the consecutive survey result data, App usage data, and applying the impact matrix. The selected App is therefore recommended to the user to improve the mental wellbeing of the user, on the prediction that the selected App will improve the numeric score in a next survey.

FIG. 9 is a block diagram representing devices, components of each device, and data flow therebetween for an app recommendation system, in accordance with some methods and embodiments of the disclosure. System 900 is shown to include a client device 918, a server 902, and a communication network 914. It is understood that while a single instance of a component may be shown and described relative to FIG. 9, additional instances of the component may be employed. For example, server 902 may include or may be incorporated in, more than one server. Similarly, communication network 914 may include or may be incorporated in, more than one communication network. Server 902 is shown communicatively coupled to client device 918 through communication network 914. While not shown in FIG. 9, server 902 may be directly communicatively coupled to client device 918, for example, in a system absent or bypassing communication network 914.

Communication network 914 may comprise one or more network systems, such as, without limitation, an Internet, LAN, WIFI, or other network systems suitable for audio processing applications. In some embodiments, system 900 excludes server 902, and functionality that would otherwise be implemented by server 902 is instead implemented by other components of system 900, such as one or more components of communication network 914. In still other embodiments, server 902 works in conjunction with one or more components of communication network 914 to implement certain functionality described herein in a distributed or cooperative manner. Similarly, in some embodiments, system 900 excludes client device 918, and functionality that would otherwise be implemented by client device 918 is instead implemented by other components of system 900, such as one or more components of communication network 914 or server 902 or a combination. In still other embodiments, client device 918 works in conjunction with one or more components of communication network 914 or server 902 to implement certain functionality described herein in a distributed or cooperative manner.

Client device 918 includes control circuitry 928, display 934, and input circuitry 916. Control circuitry 928 in turn includes transceiver circuitry 962, storage 938, and processing circuitry 940. In some embodiments, client device 918 or control circuitry 928 may be configured as client device 212 of FIG. 2.

Server 902 includes control circuitry 920 and storage 924. Each of the storages 924 and 938 may be an electronic storage device. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVRs, sometimes called personal video recorders, or PVRs), solid-state devices, quantum storage devices, gaming consoles, gaming media, or any other suitable fixed or removable storage devices, and/or any combination of the same. Each storage 924, 938 may be used to store various types of content, media data, and or other types of data (e.g., they can be used to store multimedia content such as audio, video, and advertisement data). The non-volatile memory may also be used (e.g., to launch a boot-up routine and other instructions). Cloud-based storage may be used to supplement storages 924, 938, or instead of storages 924, 938. In some embodiments, the pre-encoded or encoded multimedia content, in accordance with the present disclosure, may be stored in one or more of storages 912, 938.

In some embodiments, control circuitry 920 and/or 928 executes instructions for an application stored in memory (e.g., storage 924 and/or storage 938). Specifically, control circuitry 920 and/or 928 may be instructed by the application to perform the functions discussed herein. In some implementations, any action performed by control circuitry 920 and/or 928 may be based on instructions received from the application. For example, the application may be implemented as software or a set of executable instructions that may be stored in storage 924 and/or 938 and executed by control circuitry 920 and/or 928. In some embodiments, the application may be a client/server application where only a client application resides on client device 918, and a server application resides on server 902.

The application may be implemented using any suitable architecture. For example, it may be a stand-alone application wholly implemented on client device 918. In such an approach, instructions for the application are stored locally (e.g., in storage 938), and data for use by the application is downloaded periodically (e.g., from an out-of-band feed, from an Internet resource, or using another suitable approach). Control circuitry 928 may retrieve instructions for the application from storage 938 and process the instructions to perform the functionality described herein. Based on the processed instructions, control circuitry 928 may determine a type of action to perform in response to input received from input circuitry 916 or from the communication network 914. For example, in response to a network bandwidth maximum, control circuitry 928 may perform the steps of processes relative to various embodiments discussed herein.

In client/server-based embodiments, control circuitry 928 may include communication circuitry suitable for communicating with an application server (e.g., server 902) or other networks or servers. The instructions for carrying out the functionality described herein may be stored on the application server. Communication circuitry may include a cable modem, an Ethernet card, or a wireless modem for communication with other equipment, or any other suitable communication circuitry. Such communication may involve the Internet or any other suitable communication networks or paths (e.g., communication network 914). In another example of a client/server-based application, control circuitry 928 runs a web browser that interprets web pages provided by a remote server (e.g., server 902). For example, the remote server may store the instructions for the application in a storage device. The remote server may process the stored instructions using circuitry (e.g., control circuitry 928) and/or generate displays. Client device 918 may receive the displays generated by the remote server and may display the content of the displays locally via display 934. This way, the processing of the instructions is performed remotely (e.g., by server 902) while the resulting displays, such as the display windows described elsewhere herein, are provided locally on client device 918. Client device 918 may receive inputs from the user via input circuitry 916 and transmit those inputs to the remote server for processing and generating the corresponding displays. Alternatively, client device 918 may receive inputs from the user via input circuitry 916 and process and display the received inputs locally, by control circuitry 928 and display 934, respectively.

Server 902 and client device 918 may transmit and receive content and data such as media content via communication network 914. For example, server 902 may be a media content provider, and client device 918 may be a smart television configured to download or stream media content, such as a YouTube video, from server 902. Control circuitry 920, 928 may send and receive commands, requests, and other suitable data through communication network 914 using transceiver circuitry 960, 962, respectively. Control circuitry 920, 928 may communicate directly with each other using transceiver circuits 960, 962, respectively, avoiding communication network 914.

It is understood that client device 918 is not limited to the embodiments and methods shown and described herein. In non-limiting examples, client device 918 may be a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a handheld computer, a stationary telephone, a personal digital assistant (PDA), a mobile telephone, a portable video player, a portable music player, a portable gaming machine, a smartphone, or any other device, client equipment, or wireless device, and/or combination of the same capable of suitably displaying and manipulating media content.

Control circuitry 920 and/or 918 may be based on any suitable processing circuitry such as processing circuitry 926 and/or 940, respectively. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors, for example, multiple of the same type of processors (e.g., two Intel Core i9 processors) or multiple different processors (e.g., an Intel Core i7 processor and an Intel Core i9 processor). In some embodiments, control circuitry 920 and/or control circuitry 918 are configured to implement a media content operation system, such as systems, or parts thereof, that perform various media content manipulation processes described herein.

Client device 918 receives a user input 904 at input circuitry 916. For example, client device 918 may receive user input like a user swipe or user touch, as previously discussed. In some embodiments, client device 918 is a media device (or player), with the capability to access media content. It is understood that client device 918 is not limited to the embodiments and methods shown and described herein. In non-limiting examples, client device 918 may be a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a handheld computer, a stationary telephone, a personal digital assistant (PDA), a mobile telephone, a portable video player, a portable music player, a portable gaming machine, a smartphone, or any other television equipment, computing equipment, or wireless device, and/or combination of the same.

User input 904 may be received from a user selection-capturing interface that is separate from device 918, such as a remote-control device, trackpad, or any other suitable user movement sensitive or capture devices, or as part of device 918, such as a touchscreen of display 934. Transmission of user input 904 to client device 918 may be accomplished using a wired connection, such as an audio cable, USB cable, ethernet cable, or the like attached to a corresponding input port at a local device, or may be accomplished using a wireless connection, such as Bluetooth, WIFI, WiMAX, Zigbee, GSM, UTMS, CDMA, TDMA, 3G, 4G, 4G LTE, or any other suitable wireless transmission protocol. Input circuitry 916 may comprise a physical input port such as a 3.5 mm audio jack, RCA audio jack, USB port, ethernet port, or any other suitable connection for receiving audio over a wired connection, or may comprise a wireless receiver configured to receive data via Bluetooth, WIFI, WiMAX, GSM, UTMS, CDMA, TDMA, 3G, 4G, 4G LTE, or other wireless transmission protocols.

Processing circuitry 940 may receive input 904 from input circuit 916. Processing circuitry 940 may convert or translate the received user input 904 that may be in the form of gestures or movement to digital signals. In some embodiments, input circuit 916 performs the translation to digital signals. In some embodiments, processing circuitry 940 (or processing circuitry 926, as the case may be) carries out disclosed processes and methods.

The systems and processes discussed above are intended to be illustrative and not limiting. One skilled in the art would appreciate that the actions of the processes discussed herein may be omitted, modified, combined, and/or rearranged, and any additional actions may be performed without departing from the scope of the invention. More generally, the above disclosure is meant to be exemplary and not limiting. Only the claims that follow are meant to set bounds as to what the present disclosure includes. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real-time. It should also be noted that the systems and/or methods described above may be applied to, or used in accordance with, other systems and/or methods. In this specification, the following terms may be understood in view of the below explanations:

All of the features disclosed in this specification (including any accompanying claims, abstract, and drawings), and/or all of the steps of any method or process so disclosed, may be combined in any combination, except combinations where at least some of such features and/or steps are mutually exclusive.

Each feature disclosed in this specification (including any accompanying claims, abstract, and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed. The claims should not be construed to cover merely the foregoing embodiments, but also any embodiments which fall within the scope of the claims.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

The readers attention is directed to all papers and documents which are filed concurrently with this specification in connection with this application and which are open to public inspection with this specification, and the contents of all such papers and documents are incorporated herein by reference.

1. A method for providing an application recommendation, the method comprising:
sending a first survey to a user's device;
receiving usage statistics of a plurality of applications on the user's device;
receiving first result data of the survey; and
recommending an application based on the result data and usage statistics.

2. The method of claim 1, the method further comprising calculating an impact score for each application of the plurality of applications.

3. The method of claim 2, wherein the impact score represents an impact that each application of the plurality of applications has on the survey result data.

4. The method of claim 2, wherein the impact score is calculated according to the equation:
$${I}_{asq} = \frac{{H}_{a s}}{{\sum }_{a} {H}_{as}} {Δ}_{sq}$$
where I_{asq} represents the impact score of an application, a, on a question, q, during a survey period, s;
where Hₐₛ represents hours of usage of the application, a, during the survey period, s;
where Δ_{sq} represents a change in a survey question score during the survey period, s.

5. The method of claim 2, the method further comprising building an impact matrix based on the calculated impact score.

6. The method of claim 5, wherein the impact matrix represents an average impact of each application of the plurality of applications.

7. The method of claim 6, wherein the impact matrix is defined by:
$${I}_{aq} = \frac{1}{S} {\sum }_{S = 1}^{S} {I}_{asq}$$
where S represents a total number of surveys
where I_{aq} represents an impact of an application, a, on question, q.

8. The method of claim 5, the method further comprising sending the calculated impact score and impact matrix to a data storage.

9. The method of claim 5, the method further comprising processing the usage statistics and survey result data with the impact matrix to generate the recommendation of an application.

10. The method of claim 1, further comprising initiating a wait period.

11. The method of claim 10, wherein the wait period is initiated in response to:
the first survey being completed;
the first result data being sent to data storage; or
in response to the user not opening the first survey.

12. The method of claim 1, the method further comprising sending a second survey.

13. The method of claim 12, wherein the second survey is sent after the wait period ends.

14. The method of claim 1, wherein the survey is accessed through a URL sent to the user device.

15. The method of claim 1, the method further comprising:
requesting permission to access app usage statistics; and
in response to being granted permission:
sending app usage statistics to data storage;
in response to being denied permission:
waiting for permission to be granted before accessing app usage statistics.

16. The method of claim 1, wherein the recommendation of an application is made to improve at least one of the user's:
mental health and wellbeing;
chronic pain;
musculoskeletal conditions;
diabetes management; or
birth control management.

17. The method of claim 1, wherein if the first result data of the survey exceeds a threshold, the method further comprises at least one of:
labelling a user for clinical intervention; and
initiating an emergency response procedure.

18. A system for providing an application recommendation, the system comprising:
a data storage;
a survey delivery apparatus;
a user device; and
control circuitry configured to carry out the method of claim 1.

19. A non-transitory machine-readable medium comprising memory with instructions encoded thereon for providing an application recommendation, comprising instructions for carrying out the method of claim 1.

20. An apparatus for providing an application recommendation, the apparatus comprising means for carrying out the method of claim 1.
